(19) United States
(12) Patent Application Publication
(10) Pub. No.: US 2012/0045479 A1
(43) Pub. Date: Feb. 23, 2012

(54) Dry Powder Formulations, Vaccines and Methods

(76) Inventors: Robert E. Sievers, Boulder, CO (US); Robert L. Garcea, Boulder, CO (US); Stephen P. Cape, Boulder, CO (US)

(21) Appl. No.: 13/124,701

(22) PCT Filed: Oct. 15, 2009
(86) PCT No.: PCT/US2009/060794

§ 371 Date: Nov. 10, 2011

Related U.S. Application Data
(60) Provisional application No. 61/106,550, filed on Oct. 17, 2008.

(51) Int. Cl. A61K 39/12; A61K 47/18; A61P 31/20; A61K 9/14

(52) U.S. Cl. 424/400; 424/204.1; 424/192.1; 424/278.1

(57) ABSTRACT

Respirable dry powder formulations comprise myo-inositol and leucine. Dry powder human papillomavirus (HPV) vaccine formulations comprise a least one HPV capsid protein and a carrier comprising myo-inositol and leucine. Methods of administering an HPV vaccine to an individual comprise inhalation administration to the individual of a dry powder HPV vaccine formulation comprising a least one HPV capsid protein and a carrier comprising myo-inositol and leucine.

### FIELD OF THE INVENTION

The present invention is directed to respirable dry powder formulations, dry powder vaccine formulations, including human papillomavirus (HPV) vaccine formulations, methods of forming dry powder formulations, and methods of administering an HPV vaccine to an individual.

### BACKGROUND OF THE INVENTION

Human Papillomavirus (HPV) is the most prevalent sexually transmitted disease in the United States. Some studies have reported that approximately 20 million Americans are currently infected and over 6 million more are infected each year, that fifty percent of sexually active men and women are infected with HPV at some time in their lives, and that by the age of 50, eighty percent of sexually active women have been infected with at least one variety of HPV. Over 100 serotypes of HPV have been identified. Infections may be asymptomatic or may cause warts and/or lesions or even lead to cervical, penile, anal or throat/neck cancer. HPV is the primary cause of cervical cancer and the second leading cause of cancer deaths in all women. In the United States, 10,000 women are diagnosed with and 4,000 women die of cervical cancer every year. Worldwide, there are half a million cases of cervical cancer, leading to approximately one quarter of a million deaths yearly. Additionally, HPV has been found in up to 60% of neck and head cancer samples.

In recent years, effective but expensive vaccines for HPV such as Gardasil® and Cervarix® have gained approval in the U.S. and abroad. These vaccines use recombinant self assembled virus-like particles (VLPs) made up of 72 μl capsid protein pentamers which self-assemble to form virus-like spheres without the L2 capsid protein and viral genome, thus providing no risk of infection. Gardasil® uses recombinant self assembled VLPs made up of HPV L1 capsid proteins of the HPV types 16 and 18 (which cause 70% of cervical cancer) and 11 and 6 (which cause 90% of genital warts). Gardasil® is recommended for girls aged 11 to 26, although it may be given to girls as young as 9. The vaccine is recommended for administration by injection in a 3-dose series over 6 months in order to give maximum protection. Cervarix® contains HPV types 16 and 18 and approval is pending in the United States.

Unfortunately, however, the current HPV vaccines are only prophylactic and, although they may improve recovery from current HPV outbreaks, they are not considered therapeutic. Additionally, the cost of a current HPV vaccine series may often be considered prohibitive, both in the U.S. and abroad. The extended vaccination series requiring three doses may often be problematic, particularly in areas without consistent healthcare where women may not be able to access a health-care worker over the required 6 months. Further, the injection administration, particularly in a three dose series, increases the dangers of needle reuse, the spread of blood borne disease, the cost of safe disposal of syringes, and the need for skilled health care providers for vaccine administration.

Accordingly, while the current HPV vaccines provide a significant improvement for protecting against the most prevalent forms of HPV, further improvements are desired.

### SUMMARY OF THE INVENTION

Accordingly, the present invention is directed to respirable dry powder formulations, dry powder vaccine formulations, methods of administering an HPV vaccine to an individual, and methods of forming a respirable dry powder formulation.

In one embodiment, the invention is directed to a dry powder human papillomavirus (HPV) vaccine formulation comprising a least one HPV L1 capsid protein and a carrier comprising myo-inositol and leucine.

In another embodiment, the invention is direct to a method of administering a human papillomavirus (HPV) vaccine to an individual, comprising inhalation administration to the individual of a dry powder HPV vaccine formulation comprising a least one HPV L1 capsid protein and a carrier comprising myo-inositol and leucine.

In another embodiment, the invention is directed to a respirable dry powder formulation comprising myo-inositol and leucine. In yet another embodiment, the invention is directed to a method of forming a respirable dry powder formulation, comprising rapidly expanding a pressurized aqueous mixture of myo-inositol, leucine and carbon dioxide from a low volume mixing area through a restrictor, and drying resulting fine droplets to form the dry powder formulation.

Various advantages of the respirable dry powder formulations, the dry powder HPV vaccine formulations, the methods of administration and/or the methods of forming respirable dry powder formulations of the invention over the prior art are apparent from the following detailed disclosure.

### BRIEF DESCRIPTION OF THE DRAWINGS

The various aspects of the invention and selected embodiments thereof are more fully understood in view of the drawings, in which:

FIG. 1 is a scanning electron micrograph (SEM) of a respirable dry powder formulation according to the invention;

FIG. 2 is a SEM of a comparative dry powder formulation; and

FIGS. 3A and 3B are SEMs of a respirable dry powder vaccine formulation according to the invention.

### DETAILED DESCRIPTION

In one aspect, the present invention is directed to respirable dry powder formulations comprising myo-inositol and leucine. The combination of myo-inositol and leucine provides dry powder formulations of good stability and desirable size for pulmonary delivery by inhalation, particularly when the dry powder is formed by carbon dioxide-assisted nebulization as described herein. More specifically, myo-inositol, also known historically as “meat sugar” or cis-1,2,3,5-trans-4,6-cyclohexanehexyl, is a six carbon sugar alcohol that is found in nature and is also naturally present in human blood and body fluids. Free myo-inositol has extremely low toxicity and may be derived, for example, from rice. While sorbitol has traditionally been used as a carrier in pharmaceutical formulations such as vaccines, sorbitol tends to be sticky and difficult to disperse and tends to pick up water when exposed to moisture. Conversely, myo-inositol is less hygroscopic than most sugar excipients. Myo-inositol also exhibits a high glass transition temperature and good solubility and is not a reducing sugar. However, nebulization and drying of pure myo-inositol from an aqueous solution typically results in crystalline powders having very low fine particle fractions (FPF) and exhibiting particle aggregation, even using various solution concentrations, flow rates, drying times, and the like. Therefore, the use of myo-inositol alone as a carrier is typically undesirable for preparing formulations for pulmonary delivery. Surprisingly, the use of a combination of a small amount of leucine with the myo-inositol in the dry powder formulation forms particles having good stability and desirable sizes for pulmonary delivery.

The amount of leucine which is combined with the myo-inositol in the respirable dry powder formulations of the invention is that which is effective to increase the FPF content, and, in one embodiment, to particularly increase the content of particles having a size less than about 6 μm in aerodynamic diameter, and/or reduce the aggregation of particles, as compared with the use of myo-inositol alone. In a specific embodiment, the dry powder formulation comprises from about 90 to about 99.9 weight percent myo-inositol and from about 0.01 to about 10 weight percent leucine, based on the combined weight of myo-inositol and leucine. In a more specific embodiment, the respirable dry powder formulation comprises from about 95 to about 99 weight percent myo-inositol and from about 1 to about 5 weight percent leucine, based on the combined weight of myo-inositol and leucine. In a yet more specific embodiment, the respirable dry powder formulation comprises from about 98 to about 99 weight percent myo-inositol and from about 1 to about 2 weight percent leucine, based on the combined weight of myo-inositol and leucine.

The respirable dry powder formulations of the invention comprise FPFs allowing pulmonary delivery of particles by inhalation. In specific embodiments, respirable dry powder formulations have a fine particle fraction of from about 1 to about 10 μm in aerodynamic diameter, and in a more specific embodiment, the respirable dry powder formulations have a fine particle fraction of less than 6 μm in aerodynamic diameter. Reference to particle size by aerodynamic diameter herein refers to measurement with an Andersen Cascade Impacter. As demonstrated in the examples below, the dry powder formulations have, in certain embodiments, a fine particle fraction of less than 3.3 μm in aerodynamic diameter, sizes most effective for alveolar deposition. Particles less than about 5.8 μm in aerodynamic typically coat the entire respiratory tract, including the trachea and nasal passages. Thus, the dry powder formulations provide simple and safe formulations for pulmonary delivery specifically, and the entire respiratory tract. In a specific embodiment, the respirable dry powder formulation comprises particles having a fine particle fraction of 3% or more by weight or by volume, of a size of less than 5.8 μm in aerodynamic diameter. In another embodiment, the respirable dry powder formulation comprises particles having fine particle fractions of at least 1.4% of a size less than 3.3 μm and at least 3.7% of a size less than 5.8 μm, in aerodynamic diameter.

The dry powder formulations can be used as a respirable placebo for other pharmaceutical powders, for pulmonary delivery of myo-inositol for therapeutic purposes, for example for decreasing blood pressure or shrinking lung lesions in smokers, and/or as a carrier for other pharmaceutical active ingredients. For example, the myo-inositol can act as a carrier or bulking agent for a pharmaceutical active ingredient. In one embodiment, the dry powder formulations are used as a carrier for a vaccine. In a specific embodiment of such formulations, the vaccine is an HPV vaccine.

More specifically, in one embodiment, the dry powder formulations comprise dry powder HPV vaccine formulations comprising a least one HPV L1 capsid protein and a carrier comprising myo-inositol and leucine. HPV capsid L1 proteins are known in the art and are disclosed, for example, in U.S. Pat. Nos. 6,165,471 to Garcea et al and 7,279,306 to Schlegel et al, both of which are incorporated herein by reference. As disclosed by Garcea et al and Schlegel et al, a capsid protein is the structural protein of a virus, e.g., enveloped or non-enveloped, which constitutes the capsid structure. The L1 protein is the structural protein of papillomavirus (PV) which constitutes the major portion of the PV capsid structure. Capsid proteins polymerize to form capsomeres, the structure that makes up the larger viral capsid structure. In the case of PV, a native capsomere comprises a pentamer of L1 capsid proteins. The capsid or capsid structure refers to the structural portion of a virus that is comprised of capsomeres. In the case of PV, the viral capsid is comprised of 72 capsomeres.

In the vaccine formulations, of the invention, the individual HPV L1 capsid proteins that are employed have been altered to prevent self-assembly as VLPs, as taught by Garcea et al and Schlegel et al, and present at least one virus-neutralizing conformational epitope of L1 expressed by a native (wild type infectious) HPV virus. The HPV L1 capsid proteins have potent immunogenicity and are stable for use in vaccines. As taught by Garcea et al and Schlegel et al, the HPV L1 capsid proteins are produced recombinantly from HPV L1 DNA. In accordance with the invention, the subject capsid proteins may be produced using any desired HPV L1 DNA. In specific embodiments, the HPV L1 DNA is derived from an HPV which is involved in cancer or condylomata acuminata, e.g., HPV-16, HPV-18, HPV-31, HPV-33, HPV-35, HPV-39, HPV-45, HPV-51, HPV-52, and HPV-56 are involved in cancer, and HPV-6, HPV-11, HPV-30, HPV-42, HPV-43, HPV-44, HPV-54, HPV-55, and HPV-70, are involved in warts. In specific embodiments, the HPV L1 capsid protein comprises HPV 16, HPV 18, HPV 11, or HPV 6. In another embodiment, the vaccine formulation comprises two or more HPV L1 capsid proteins, i.e., combinations of two or more L1 capsid proteins of HPV 16, HPV 18, HPV 11, and HPV 6, or other of the noted HPV L1 capsid proteins. The HPV L1 capsid proteins are economically produced from E. coli and are very stable. The HPV L1 capsid proteins have also been shown to cause a strong cytotoxic T-cell response, indicating that a therapeutic as well as prophylactic effect may be provided.

In further embodiments, the HPV L1 capsid protein may comprise a fusion protein, and in a specific embodiment may comprise an HPV L1 capsid protein fused to glutathione-5-transferese (GST). As taught by Schlegel et al, the L1 capsid protein may be fused to GST at its amino or carboxy terminus. In a more specific embodiment, the HPV L1 capsid protein is HPV 16 L1 capsid protein fused to GST. Alternatively, the HPV L1 protein may be trypsinized as taught by Li et al, Journal of Virology, 71(4):2988-2995 (1997), also incorporated herein by reference, wherein the L1 capsid protein is digested with trypsin. In a specific embodiment, the HPV L1 capsid protein is trypsinized HPV 11 L1 capsid protein.

The dry powder HPV vaccine formulation as described comprises an immunity effecting amount of the HPV L1 capsid protein and an amount of carrier sufficient to allow dry particle formation. In one embodiment, the dry powder HPV vaccine formulation comprises from about 0.01 to about 100 μg HPV L1 capsid protein, per mg of the formulation, and a substantial, if not entire, balance of the carrier comprises myo-inositol and leucine. The amount of leucine which is combined with the myo-inositol in the carrier is that which is effective to increase the FPF content, and, in one embodiment, to particularly increase the content of particles having a size less than about 6 μm in aerodynamic diameter, and/or reduce the aggregation of particles, as compared with the use of myo-inositol alone as a carrier. In a specific embodiment, the carrier comprises from about 90 to about 99.9 weight percent myo-inositol and from about 0.01 to about 10 weight percent leucine, based on the weight of the carrier. In a more specific embodiment, the carrier comprises from about 95 to about 99 weight percent myo-inositol and from about 1 to about 5 weight percent leucine, based on the weight of the carrier. In yet a further embodiment the carrier comprises from about 98 to about 99 weight percent myo-inositol and from about 1 to about 2 weight percent leucine, based on the weight of the carrier.

In another aspect, the invention is directed to methods of administering an HPV vaccine to an individual. The methods comprise inhalation administration to the individual of a dry powder HPV vaccine formulation comprising a least one HPV L1 capsid protein and a carrier comprising myo-inositol and leucine. Recent research has shown that pulmonary delivered dry powder flu vaccines (influenza subunit proteins with no adjuvant) in mice induces a mucosal, systemic humoral and cell-mediated immune response superior to conventional vaccination. See, for example, Amorij, et al, Vaccine, 25(52):8707-17 (2007). Additionally, the body's lymphatic system has been shown to communicate immunity between distant lymphoid tissues in the body through the lymphatic system, leading to mucosal immunity in the genital mucosa as a result of immunization in the respiratory tract. See, for example, Holmgren et al, Nature Medicine, 11:S45-S53 (2005), and Balmelli et al, Journal of Virology, 72(10):8220-8229 (1998).

In the Balmelli study, mice given a liquid formulation of HPV-16 VLPs administered nasally under anesthetic showed a significant HPV-16-specific IgG response in the blood. High levels of HPV-16 specific IgG and IgA were also observed in the oral and vaginal mucosa when the mice were vaccinated under anesthetic, resulting in a significant deposition of the VLPs in the lungs. Thus, in the present methods, mucosal immunity of HPV will benefit the recipient by preventing initial infection and possibly acting therapeutically by aiding in viral clearance. Mucosal immunity resulting in the production of HPV targeting IgA, an antibody specific to the mucosa, inactivates the virus at the point of infection. Mucosal immunity in the airways will also reduce the risk of HPV-caused throat and neck cancer, increasing the value of this vaccine to males in which HPV-related genital cancer is rare and for whom no current vaccine is approved.

The respiratory airways provide an excellent target for vaccine delivery by inhalation. The dry powder vaccine formulations advantageously comprise a fine particle fraction of from about 1 to about 10 μm in aerodynamic diameter, and in a more specific embodiment, the dry powder vaccine formulations have a fine particle fraction of less than 6 μm in aerodynamic diameter. As demonstrated in the examples below, the dry powder vaccine formulations have, in certain embodiments, a fine particle fraction of less than 3.3 μm in aerodynamic diameter. Particles in the size range of 1-5 μm in aerodynamic diameter can deposit in the deep lung and alveolar sacs, where they are absorbed over large areas of vascularized surface. Particles in the size range of 5-10 μm will deposit at the back of the throat where they are introduced to the mucosal immune system of the tonsils. The lungs, being a natural target of microbes, contain mucosa-associated lymphoid tissues and are patrolled by dendritic cells adapted for antigen presentation to T-cells, conducive to good immune response. Particles introduced to the nasal airways will also induce an immune response in both the systemic and mucosal immune systems.

Thus, the dry powder vaccine formulations as described are advantageous in providing needle-free pulmonary delivery of the vaccine by inhalation, thereby overcoming the aforementioned disadvantages associated with current HPV vaccines requiring needle injection, including the dangers of needle reuse, the spread of blood borne disease, the cost of safe syringe disposal, and the need for skill health care administration. Additionally, as the dry powder formulations exhibit good stability, the distribution and administration of the vaccine formulations is facilitated in areas where permanent health facilities are not convenient or readily available. Further, the dosages may be varied to improve the ability to reduce the requirement for multiple inoculations to obtain the desired immunity. Thus, the ease of delivery of the dry powder vaccine formulations may increase patient compliance and overall increase efficacy.

In additional embodiments, the dry powder formulations, including the dry powder vaccine formulations, may include one or more additional excipients or carriers. In one embodiment, the dry powder formulations include a surfactant to render the powder surfaces more lipophilic. Examples of suitable surfactants include, but are not limited to lecithin and the Tween surfactants, although other surfactants will be apparent to one of ordinary skill in the art.

In another aspect, the invention is directed to methods of forming a respirable dry powder formulation. The methods comprise carbon dioxide assisted nebulization (CAN) wherein a pressurized aqueous mixture of myo-inositol, leucine and carbon dioxide is rapidly expanded from a low volume mixing area through a restrictor, and the resulting fine droplets are dried to form the dry powder formulation. The method can conveniently be conducted using a Bubble Dryer® apparatus available from Aktiv-Dry LLC of Boulder, Colo. and as described in U.S. Pat. No. 6,095,134 to Sievers et al, which is incorporated herein by reference. In a specific embodiment, the respirable dry powder formulation is formed by dissolving myo-inositol and leucine, and optionally a desired pharmaceutical active ingredient, in a fluid such as water to form a solution and mixing the solution with highly pressurized carbon dioxide, for example above or near its supercritical pressure, which becomes a gas upon rapid pressure release. The fluid is at least partially immiscible with the carbon dioxide and upon release of the pressure, the rapid expansion forms an air-borne dispersion or aerosol of droplets which are then rapidly dried to form the desired dry powder formulation. In a specific embodiment employing the Bubble Dryer® apparatus, an aqueous solution/suspension of the components is intimately mixed with carbon dioxide at 1200 psi and room temperature in a low volume tee. Upon mixing, a carbon dioxide/water emulsion is formed and the water is saturated with up to 6% (w/w) carbon dioxide. The emulsion is nebulized out of a 10 cm long, 74 micron ID silica restrictor and the force of the expanding carbon dioxide, coupled with the rapid dissolution, then expulsion of the aqueous carbon dioxide, creates a fine mist of micro- and nano-droplets. These droplets are mixed with dry nitrogen gas at 50-70° C. in a glass drying chamber and the resulting particles are collected on a filter. While other apparatus and methods may be used in preparing dry powder formulations according to the invention, the presently described process is advantageous in that it is efficient and inexpensive as compared with other known methods.

The following examples demonstrate various features of the invention.

### Example 1

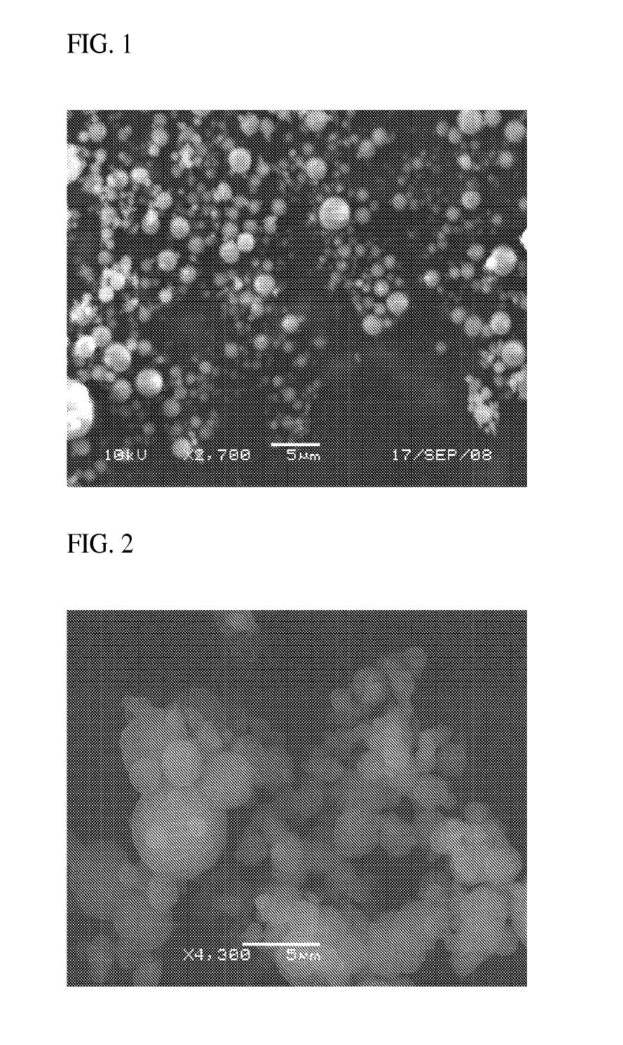

This example demonstrates the preparation of a respirable dry powder formulation according to the invention. A 10% total dissolved solids (m/v) aqueous solution containing myo-inositol and leucine at a 98.5% to 1.5% weight ratio is pressurized to 1200 psi and intimately mixed with carbon dioxide at 1200 psi in a low volume mixing tee using a Bubble Dryer® apparatus. The resulting emulsion is rapidly expanded out of the 10-cm long, 74 μm diameter restrictor to produce a plume of fine droplets and microbubbles. These droplets are quickly dried by dry nitrogen at 35 L/min and 70° C. to produce fine particles, which are collected on a filter. The resulting myo-inositol/leucine powder shows very good FPF at 19%<3.3 μm and 58%<5.8 μm in aerodynamic diameter. This powder also shows very low hygroscopicity at normal lab conditions, including a relative humidity of about 40%. A scanning electron micrograph (SEM) of this powder is set forth in FIG. 1 and shows the powder consisting of spheres approximately 0.1 to 5 μm in geometric diameter. For comparison purposes, the above described procedure is repeated while omitting the leucine component. The resulting product had very low FPFs. A SEM of the resulting product is set forth in FIG. 2 and shows agglomerated particles.

### Example 2

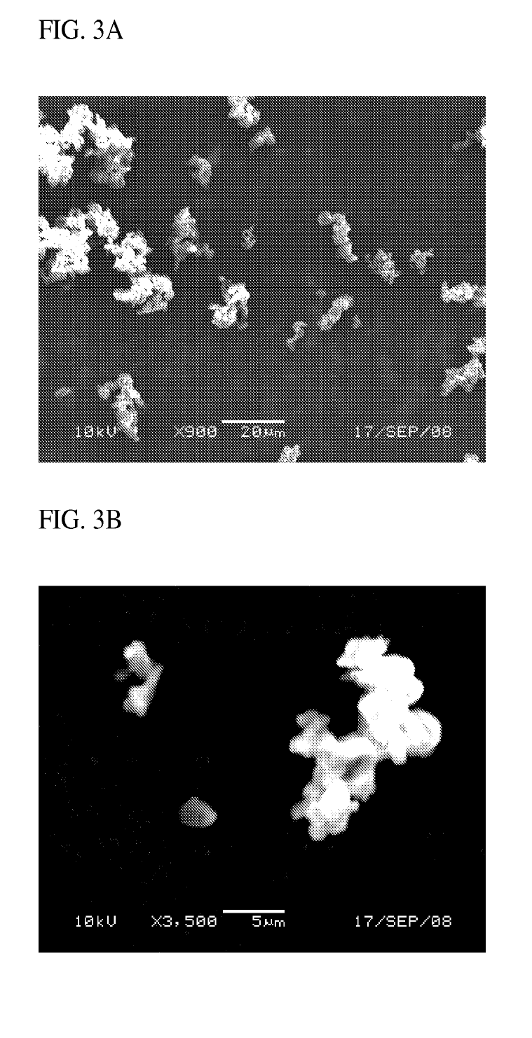

This example demonstrates the preparation of a dry powder vaccine formulation according to the invention using HPV 16 μl capsid protein fused to GST as described by Schlegel et al. The protein is provided in buffer L (40 mM Tris, 0.2 M NaCl, 2 mM DTT, 1 mM EDTA) plus 10 mM reduced glutathione at a concentration of 2.1 mg/ml. It is a fraction from the glutathione-sepharose 4B elution as described by Schlegel et al and is identified as GST-HPV 16 L1 C175S. An aqueous solution is prepared to have 10% total dissolved solids and produce dry powders that are 98.5% myo-inositol and 1.5% leucine containing 50 μg of HPV 16 capsid protein in 10 mg of final powder. This concentration is chosen to provide a dose of protein sufficient to produce an immunogenic response in 10 mg of powder, which is easily inhaled and handled. Using a Bubble Dryer® apparatus, this solution is pressurized to 1200 psi and intimately mixed with carbon dioxide at 1200 psi and room temperature in a low dead volume mixing tee. The resulting emulsion is expanded out of the 10-cm long 74-μm internal diameter restrictor into atmospheric pressure to produce a plume of micro- and nano-particles which are quickly dried by dry nitrogen gas at 35 L/min at 70° C. The resulting particles are collected on a filter and scraped off using metal spatulas. The resulting powder consists of low density, fluffy, white product. A SDS-PAGE test is conducted on the powder produced and shows that the final powder contains the HPV 16 μl capsid protein at a concentration of 1 μg/1 mg powder. The FPF of this powder was 1.4%<3.3 μm and 3.7%<5.8 μm, and with an emitted dose of 96%. SEMs of this powder are set forth in FIGS. 3A and 3B and show the powder contains micron-sized wrinkled particles arranged in loose aggregates.

The specific illustrations and embodiments described herein are exemplary only in nature and are not intended to be limiting of the invention defined by the claims. Further embodiments and examples will be apparent to one of ordinary skill in the art in view of this specification and are within the scope of the claimed invention.

What is claimed is:

1. A dry powder human papillomavirus (HPV) vaccine formulation, comprising a least one HPV L1 capsid protein and a carrier comprising myo-inositol and leucine.

2. The dry powder HPV vaccine formulation of claim 1, wherein the carrier comprises from about 90 to about 99.9 weight percent myo-inositol and from about 0.01 to about 10 weight percent leucine, based on the weight of the carrier.

3. The dry powder HPV vaccine formulation of claim 1, wherein the carrier comprises from about 95 to about 99 weight percent myo-inositol and from about 1 to about 5 weight percent leucine, based on the weight of the carrier.

4. The dry powder HPV vaccine formulation of claim 1, wherein the at least one HPV L1 capsid protein comprises an HPV L1 fusion protein.

5. The dry powder HPV vaccine formulation of claim 1, wherein the at least one HPV L1 capsid protein comprises an HPV L1 capsid protein fused to glutathione-5-transferese (GST).

6. The dry powder HPV vaccine formulation of claim 1, wherein the at least one HPV capsid protein comprises an HPV 16 μl protein fused to glutathione-5-transferese (GST).

7. The dry powder HPV vaccine formulation of claim 1, wherein the at least one HPV capsid protein comprises a trypsinized HPV L1 capsid protein.

8. The dry powder HPV vaccine formulation of claim 1, comprising from about 0.01 to about 100 μg HPV L1 capsid protein, per mg of the formulation, wherein the carrier comprises from about 95 to about 99 weight percent myo-inositol and from about 1 to about 5 weight percent leucine, based on the weight of the carrier.

9. The dry powder HPV vaccine formulation of claim 1, comprising particles of a size of from about 1 to about 10 μm in aerodynamic diameter.

10. A method of administering a human papillomavirus (HPV) vaccine to an individual, comprising inhalation administration to the individual of a dry powder HPV vaccine formulation comprising a least one HPV L1 capsid protein and a carrier comprising myo-inositol and leucine.

11. A respirable dry powder formulation comprising myo-inositol and leucine.

12. The respirable dry powder formulation of claim 11, comprising from about 90 to about 99.9 weight percent myo-inositol and from about 0.01 to about 10 weight percent leucine, based on the combined weight of myo-inositol and leucine.

13. The respirable dry powder formulation of claim 11, comprising from about 95 to about 99 weight percent myo-inositol and from about 1 to about 5 weight percent leucine, based on the combined weight of myo-inositol and leucine.

14. The respirable dry powder formulation of claim 11, comprising particles having a fine particle fraction of 3% or more less than 5.8 μm in aerodynamic diameter.

15. The respirable dry powder formulation of claim 11, further comprising a pharmaceutical active ingredient.

16. The respirable dry powder formulation of claim 15, wherein the pharmaceutical active ingredient comprises a vaccine.

17. A method of forming a respirable dry powder formulation, comprising rapidly expanding a pressurized aqueous mixture of myo-inositol, leucine and carbon dioxide from a low volume mixing area through a restrictor, and drying resulting fine droplets to form the dry powder formulation.

18. The method of claim 17, wherein the aqueous mixture further comprises a pharmaceutical active ingredient.

19. The method of claim 18, wherein the pharmaceutical active ingredient comprises a vaccine.

20. The method of claim 17, wherein the resulting dry powder formulation comprises particles of a size of from about 1 to about 10 μm in aerodynamic diameter.

21. The method of claim 17, wherein the myo-inositol acts as a carrier or bulking agent for a pharmaceutical active ingredient.
